(19) United States
(12) Patent Application Publication
(10) Pub. No.: US 2003/0166256 A1
(43) Pub. Date: Sep. 4, 2003

(54) Integrated bacteria enzyme straw/barley matrix for ponds, lakes, aquariums and aquaculture

(76) Inventors: Neal Zahn, Tulsa, OK (US); James F. Tobey JR., Salem, VA (US)

Correspondence Address: HEAD, JOHNSON & KACHIGIAN, 228 W 17TH PLACE, TULSA, OK, 74119, US

(21) Appl. No.: 10/082,849

(22) Filed: Feb. 26, 2002

(51) Int. Cl. C12N 1/20; C12N 9/14

(52) U.S. Cl. 435/252.4; 435/252.5; 435/195

(57) ABSTRACT

A fermentation composition for treatment of aquatic environments. The fermentation composition includes an activated organic matrix selected from wheat, barley or rye straw, ground, whole-grain barley grain and wheat bran. The fermentation composition also includes beneficial saprophytic bacteria, beneficial hydrolytic enzymes and soluble humatic compounds.

### BACKGROUND OF INVENTION

1. Field of the Invention

This invention relates to a product for bioaugmentation and bioremediation of aquatic environments. More specifically, this invention is directed to the use of barley straw or similar substrate which has been activated by fermentation with saprophytic bacterial organisms to release natural humatic compounds to improve the clarity and quality of water and reduce the growth of green algae and string algae in aquatic environments and to inoculate the aquatic environment with the saprophytic bacteria from the fermentation process so as to provide long term control of algae in the aquatic environment.

2. Prior Art

Decorative garden ponds are becoming very popular in the United States and around the world. While the garden pond adds a degree of beauty and tranquility to its owner's garden, it also requires regular maintenance by its owner in order to maintain its pleasing appearance. In addition to clearing leaves and other debris from the water, it is necessary to maintain the quality of the water in the pond. Among the most common water quality problems are those caused by excessive algae growth fueled by excessive nutrient levels in the water and sludge caused by the buildup of organic matter on the bottom of the pond. The most common problem algae types are those from the phyla choloophyta, euglenophyta, dinoflagellata, chrysophyta, and rhodophyta. Prokaryotes including the kingdoms monera and protists such as pediastrum, scendesmus, cosmarium, and string algae. Other problem algae include the pond scum algae such as fucus, sporiogyra, volvox, cyanbactreria and the diatoms. The above list is not all inclusive and it is understood that other species of algae like organisms are contemplated by this disclosure.

It is known in the prior art that barley straw may be added to aquatic environments so as to effect improvement in those environments through the beneficial action of the breakdown products of the straw. The application of the straw into the aqueous environment followed by natural degradative processes which occur over time reportedly releases certain beneficial humatic ingredients that positively affect the water quality, most notably the reduction in certain algal organisms, including those known as “string algae.”

The use of barley straw for this purpose suffers from several disadvantages: first, the need to use relatively large quantities of barley straw which is aesthetically unpleasing as it floats on the top of the pond and second, the process depends upon natural bacteria found in the pond to begin the breakdown of the barley which releases the natural humatic ingredients which are responsible for the beneficial activity of the straw. Because of the considerable variability in the bacterial flora found in garden ponds, the degree of effectiveness and time which it takes the barley process to function is extremely unpredictable.

The prior art also includes a process by which an aqueous extract of barley is added to ponds to prevent aquatic plant growth. Experience has shown that this product is subject to a number of disadvantages such as the contamination by natural microflora, limited shelf life of the organic compounds in the extract, and the inherent cost of conveyance of inert water component from production facility to the site of use. A description of this product can be seen in U.S. Pat. No. 6,149,929.

It is also known in the prior art to apply bacterial additives to the pond to increase the population of beneficial saprophytic bacteria in the aquatic environment. Saprophytic bacteria are those bacteria which are involved in the breakdown and decay of organic matter. The bacterial additives also contain beneficial enzymes produced as a byproduct of bacterial metabolism. The saprophytic bacteria break down organic waste and nutrients that normally feed the growth of green algae. The bacterial preparations have demonstrated effectiveness in combating green algae, the cause of green water in ponds, however, the bacterial preparations have been largely ineffective against string algae which builds up primarily in shallow places such as streams and waterfalls. Examples of bacterial additives seen in the prior art can be found for example in the Winston Company product CRYSTAL CLEAR® (http://www.winstoncompany.com/cc/products/claritymax.asp).

For the foregoing reasons there is a need for a biological water treatment which is effective against both green water algae and string algae buildup in ponds, lagoons, aquariums, aquaculture systems, waste water treatment, holding or conveying systems which may be produced under controlled conditions.

### SUMMARY OF THE INVENTION

The present invention is directed to a composition that satisfies the need for a biological water treatment which is effective against both green water algae and string algae buildup in ponds, lagoons, aquariums, aquaculture systems, waste water treatment, holding or conveying systems and to the process by which that composition is made.

A composition having features of the present invention comprises an organic matrix of straw or grain, the matrix being inoculated with certain strains of beneficial saprophytic bacteria and fermented under controlled conditions. The saprophytic bacteria activates the matrix during the fermentation process thereby releasing beneficial humatic compounds which have a positive effect on water quality in aquatic environments as well as producing hydrolytic enzymes which help to break down nutrients in the water which feed the algae and inoculating the water with a colony of beneficial saprophytic bacteria which remain viable and contribute to the long term effectiveness of the water treatment. These and other features, aspects, and advantages of the present invention will become better understood with reference to the following description and appended claims.

### DETAILED DESCRIPTION OF THE PREFERRED EMBODIMENTS

The composition of the present invention comprises a matrix of organic material such as grain or straw or similar material upon which an inoculum of beneficial saprophytic bacteria have been fermented under controlled conditions of temperature and humidity. The fermenting process activates the organic matrix, releasing beneficial hydrolytic enzymes and soluble humatic compounds which positively affect water quality as well as inoculating the aquatic environment with live beneficial saprophytic bacteria which will continue to be active for a long period of time.

In one preferred mode, the present invention may be made by a process by which an organic matrix of straw material to which essential bacterial nutrients have been added is first steam pasteurized and then inoculated with a culture of selected saprophytic bacteria. After the organic matrix is inoculated with the bacteria culture, it is incubated under controlled temperature and moisture conditions to allow the culture to grow and to allow the bacteria to predigest the organic matrix. This step activates the organic matrix releasing beneficial humatic compounds which are the by-product of the breakdown of the organic matrix. The bacteria secrete hydrolytic enzymes which have a beneficial effect. After the incubation step, the temperature is increased and the humidity is decreased to dry out the organic matrix and cause the bacteria to become dormant. The dried product is then ground through a hammermill to create a granulated final product.

In the first embodiment, the composition of this invention is made by growing specific saprophytic strains of bacteria in barley straw and certain micronutrients. A second embodiment is made by growing the aforementioned strains of bacteria on a substrate matrix composed of one or more of the following: wheat, barley or rye straw, ground, whole-grain barley grain and wheat bran. A third embodiment is made by growing the aforementioned strains of bacteria on a substrate matrix composed of wheat bran and ground, whole-grain barley. Another embodiment combines elements of the three. The bioremediative composition of the present invention is preferably made by incorporating bacteria/enzyme combinations and other biologically active organic materials in a matrix in order to create a composition with bioremediative components including viable bacteria, enzymes, and other beneficial compounds such as soluble humatic breakdown products of barley and barley straw into an aqueous environment. Instead of the combination, bacteria or enzymes can be admixed and thus incorporated into the straw matrix separately, but this is not preferred. Enzymes produced by the bacteria on the straw/grain product matrix, along with the humatic or other catabolic breakdown products of the straw, initiate beneficial action in the aquatic system to which they are applied, whereas the bacteria become a permanent digestive colony in the aquatic system.

The purpose of said invention is to provide an integrated bioremediative product with ready-to-use, active biological compounds derived from specific types of straw and grains along with viable bacteria and their hydrolytic enzymes which through their beneficial effects would render aquatic environments such as, but not limited to, ponds, lagoons, aquariums, aquaculture systems, waste water treatment, holding or conveying systems more aesthetically pleasing, efficient in aquatic animal production, and less susceptible to algae and other undesirable aquatic plants, thus requiring less maintenance.

The composition of the present invention may be made by the following process. Specific saprophytic bacteria are selected and produced using any of several common microbiological techniques in suitable aqueous substrates in order to form an inoculum which composes the active microbial seed for final product fermentation. This active microbial seed or inoculum is mixed with a substrate composed of steam pasteurized barley and/or grain straw. In order to reduce the difficulty of mechanical handling, the straw may be partially ground or comminuted into pieces which are from 0.2 cm to 5 cm in length. Water is added to the straw, ideally in the amount of 35-60% by weight, with the optimum amount being in the range of 40-55% based upon the weight of the total composition.

Other nutrients which accelerate the growth of degradative bacteria may optionally be added to the substrate. These nutrients may include nitrogen-containing compounds such as urea, ammonium sulfate, or protein hydrolysates. In addition, buffering salts such as calcium carbonate or sodium bicarbonate are added to help stabilize the pH of the matrix. The pH in the 6.0-8.0 range is desirable. This allows the bacterial seed to attain rapid and consistent growth rates. Small amounts of other micronutrients may be required or desirable depending upon the choice of bacteria selected as an inoculum.

The moist matrix composed of straw, water, buffer and micronutrients is subjected to heat so that the matrix is raised to at least 100° Celsius for a minimum of 20-45 minutes. Ideally, the mixture may be subjected to positive pressure and temperatures up to 121° Celsius for the time of pasteurization. This process reduces the level of indigenous microbial contaminants and renders the straw more receptive to digestion by the added bacterial inoculum. After cooling the straw substrate to a temperature below the thermal death point of the inoculated bacteria, the seed inoculum is mixed with the straw substrate. The final moisture content may be in the amount of 35-60% by weight, based upon the weight of the total composition.

The mixture is then allowed to incubate for approximately 18-30 hours. During this time, the bacterial inocula grow, multiply and produce degradative enzymes which partially digest the non-cellulosic components of the straw substrate and solubilize certain organic humatic components that are desirable for aquatic addition. The incubation step may be accomplished in various ways, including the following examples, which are not to be construed as limitations of this invention.

### EXAMPLE 1

A dry, straw organic matrix substrate was prepared using the following ingredients:

| Ingredient | Percent by weight |
|---|---|
| Dry barley straw (ground to 1.0-3.0 cm length) | 97.9 |
| Calcium carbonate | 1 |
| Urea | 0.5 |
| Brewers Yeast | 0.5 |
| Monopotassium phosphate | 0.1 |

The dry organic matrix substrate was initially mixed by mechanical means with approximately 1 part of water per 2 parts substrate by weight and conveyed by screw conveyer to stainless steel cookers with augers for material mixing and movement, which are equipped with live steam injection nozzles. This initial mixing is performed to moisten the mixture.

The substrate was next pasteurized at 100-105° Celsius in the presence of saturated steam for 30-40 minutes. During this time, the moisture content of the substrate was increased to approximately 40% by weight. The substrate was then cooled by a moving flow of cool filtered air until the temperature of the substrate is less than 45° Celsius. Next, a previously prepared liquid inoculum of saprophytic bacteria selected from the spore-forming Bacillus genus including one or more strains selected from a group consisting of Bacillus subtilis, Bacillus licheniformis, Bacillus amyloliquefaciens, Paenibacillus polymyxa, Bacillus megaterium, Bacillus psychrophilus, Bacillus globiformis, Bacillus psychrosaccharolyticus, Bacillus benzoevorans, Bacillus vallismortis, Bacillus mojavensis, Bacillus stearothermophilus, and Bacillus acidopullyticus was blended onto the substrate. At this time, the moisture level of the inoculated substrate will be in the 48-55% by weight range based upon the weight of the total composition.

Next, the inoculated straw substrate was placed upon perforated stainless steel or aluminum trays (according to the process such as disclosed in Jeffreys, U.S. Pat. No. 4,055,666) in layers approximately 4-5 cm in thickness. The trays are placed upon racks equipped with rollers that allow conveyance into a temperature and humidity controlled chamber. The inoculated straw substrate is confined in said environmentally controlled chamber for about 30 hours under high humidity conditions. During this time period, the bacterial inoculum will grow to approximately 2×10⁹ colony forming units/gram (CFU/g); hydrolytic enzymes are produced and excreted by the bacteria and hydrolytic digestion and solubilization of the non-cellulosic humatic components of the straw occurs. At that time, the humidity is reduced and the temperature increased to 40° Celsius. At this temperature, under low humidity, the solubilized humatic components of the straw, the straw substrate, and the beneficial bioaugmentative bacteria are immobilized and attached to the straw substrate and dehydrated to 12% or less moisture content based upon the weight of the total composition. Other procedures to grow the bacterial inoculum and hydrolytic enzymes might also be employed.

The dried material which results from this process is composed of the cellulosic remaining matrix of the straw substrate, the viable bioaugmentative saprophytic bacteria, the hydrolytic enzymes produced by said bacteria, and the hydrolyzed and solubilized humatic components of the straw substrate in an integral product. The dried product may then be ground through a hammermill with 2/64 inch screen, resulting in comminution of the integrated product into a granular, homogenous powder. The dry granular material may be packaged, stored, shipped and used in pond or other aquatic bioaugmentation processes.

### EXAMPLE 2

A dry substrate was prepared using the following ingredients:

| Ingredient | Percent by weight |
|---|---|
| Wheat Bran | 72.9 |
| Dry whole barley grain (ground to 1 mm in diameter) | 25 |
| Calcium carbonate | 1 |
| Urea | 0.5 |
| Brewers Yeast | 0.5 |
| Monopotassium phosphate | 0.1 |

The dry substrate was blended, mixed with water and processed in similar fashion to that process described in Example 1 to yield a dry, granular material integrating the components of straw and to immobilize the bioaugmentative bacteria in same material.

### EXAMPLE 3

A dry substrate was prepared using the following ingredients:

| Ingredient | Percent by weight |
|---|---|
| Wheat Straw | 25 |
| Barley Straw | 25 |
| Wheat Bran | 25 |
| Dry whole barley grain (ground to 1 mm in diameter) | 22.9 |
| Calcium carbonate | 1 |
| Urea | 0.5 |
| Brewers Yeast | 0.5 |
| Monopotassium phosphate | 0.1 |

The substrate was blended, mixed with water and processed in identical fashion to that process described in Example 1 to yield a dry, granular material integrating the components of straw and to immobilize the bioaugmentative bacteria in same material.

Usage of the present invention may be observed from the following examples.

### EXAMPLE 4

An ornamental pond in Naples, Fla. presented a problematic situation that was typical of water gardens and ornamental ponds. The water itself was extremely clear, but string algae were not of the species volvox, spirogyra and fucus. The managers of the pond applied barley straw according to the teaching of Ben Helm Koi Ponds and Garden's article of August 2001, page 35. The barley straw made no apparent difference in the quantity of string algal growth or in the physical nature of this contaminating organism. In addition, the pond was treated for four weeks with a commercial bioaugmentation agent comprised of naturally occurring bacteria and hydrolytic enzymes. Upon testing the water in this pond, it was found that the water was neutral in pH and had no significant levels of soluble ammonia (NH3), Nitrite (—NO2—) and Nitrate (—NO3-2). At the end of this four week treatment, once again, there was no apparent difference in the quantity of string algal growth or in the physical nature of this contaminating organism. The pond was treated at the rate of one tablespoon per gallon for every 100 gallons of water in the pond with product prepared utilizing the same formula as stated above of commercial bioaugmentation agent comprised of naturally occurring bacteria and hydrolytic enzymes with the following variation: 22% of the inert ingredients in the formula were replaced with product prepared according to the process outlined in Example 2. One week after treating the pond with this combination product (pretreated/fermented barley containing live microbes and their indigenous enzymes), it was observed that the string algae had begun to deteriorate, showing extensive changes in its physical character (the first stages of the start of the removal of algae in the system).

### EXAMPLE 5

An ornamental pond in Bonita Springs, Fla. experienced a common problem in this water environment. There was a measurable nitrate (NO3-2) level (18 ppm) and a high Green Algae population single cells diatoms. The pond observed in this example was a newly constructed pond only 4 weeks old. It is common at this stage to experience a severe green water break-in period. It is also common to use a bacteria/enzyme containing product to hasten the maturation of the pond in such manner as to eliminate the green algae problem, such as a commercial bioaugmentation agent, for example Aquascapes—Liquid AquaClear®, an agent comprised of naturally occurring bacteria and hydrolytic enzymes, according to label directions of one ounce per 1000 gallons of water one time per week for the four week period. The use of this product alone, however, had not corrected the green algal contamination problem to date. The use of this product was then discontinued. An activated barley formulation (in this example, commercially available AquaClear®) containing 70% by weight of the activated barley product prepared according to Example 4 (above) was used to treat the pond. Two days after initiating treatment, nitrate levels decreased 20% as shown in Table 1 (below). After four days of treatment, nitrate levels decreased 66.7% and water clarity as measured by visibility increased to 2.5 feet allowing the bottom of pond to be seen. It was visually apparent that the green, single-celled algae had drastically decreased and was being controlled as result of the treatment.

TABLE 1
TREATMENT OF BONITA SPRINGS, FLORIDA POND

| Time | Nitrate Level | | Visibility | |
|---|---|---|---|---|
| (in days after treatment) | (NO3-2) | | (feet) | Algae Control |
| 0 | 18 | ppm | | None |
| 2 | 15 | ppm | | Some action |
| 4 | 5 | ppm | 2.5 feet+ | Positive control |

Changes may be made in the construction and the operation of various components, elements, and assemblies described herein or in the steps or the sequence of steps of the methods described herein without departing from the spirit and scope of the invention as defined in the following claims.

What is claimed is:

1. A fermentation composition for treatment of aquatic environments, the composition comprising:
an activated organic matrix, beneficial saprophytic bacteria, beneficial hydrolytic enzymes, and soluble humatic compounds.

2. The composition according to claim 1 wherein the activated organic matrix is comprised of one or more products selected from a group consisting of wheat, barley or rye straw, ground, whole-grain barley grain and wheat bran.

3. The composition according to claim 1 wherein said beneficial saprophytic bacteria are composed of one or more strains selected from the group consisting of Bacillus subtilis, Bacillus licheniformis, Bacillus amyloliquefaciens, Paenibacillus polymyxa, Bacillus megaterium, Bacillus psychrophilus, Bacillus globiformis, Bacillus psychrosaccharolyticus, Bacillus benzovorans, Bacillus vallismortis, Bacillus mojavensis, Bacillus stearothermophilus, and Bacillus acidopullyticus.

4. The composition according to claim 1 wherein the organic matrix is activated by fermentation in the presence of beneficial saprophytic bacteria.

5. The composition according to claim 1 wherein the hydrolytic enzymes are produced during the fermentation of the organic matrix by the beneficial saprophytic bacteria.

6. The composition according to claim 1 wherein the soluble humatic compounds are produced by the fermentation of the organic matrix by the beneficial saprophytic bacteria.

7. The composition according to claim 1 wherein the organic matrix is comprised of from 10% to 75% wheat straw and wheat bran.

8. The composition according to claim 1 wherein the organic matrix is comprised of from 10% to 75% other straw or grain products.

9. The composition according to claim 1 wherein the organic matrix is comprised of from 10% to 98% barley and/or grain.

10. The composition according to claim 1 wherein the composition is a dry granulated fermentation product.

11. A method for producing a dried granular fermentation product for the treatment of aquatic environments comprising the following steps:
(a) providing an organic matrix;
(b) adding water in the amount of 35% to 60% by weight based on the weight of the total composition to said organic matrix;
(c) steam pasteurizing the organic matrix;
(d) inoculating the pasteurized organic matrix with seed bacterium;
(e) incubating the organic matrix until bacterial growth occurs; and
(f) drying the organic matrix to immobilize the saprophytic bacteria.

12. A method as set forth in claim 11 including the additional step of chopping said organic matrix into pieces from about 0.2 cm to about 5 cm in length prior to said addition of water.

13. A method as set forth in claim 11 including the additional steps of adding additional nutrients to said organic matrix to accelerate growth of bacteria and adding buffering salts to the organic matrix to control pH for optimum bacterial growth prior to stream pasteurization.

14. A method as set forth in claim 11 including the additional step of grinding the organic matrix after said drying to create a dried granular fermentation product.

15. A method for treating an aquatic environment comprising the steps of:
adding a fermentation composition of an actuated organic matrix, beneficial saprophytic bacteria, beneficial hydrolytic enzymes, and soluble humatic compounds to the aquatic environment in an amount sufficient to reduce growth of algae in the aquatic environment.
